(19) United States
(12) Patent Application Publication
(10) Pub. No.: US 2024/0342403 A1
(43) Pub. Date: Oct. 17, 2024

(54) The Overdose Reviver Automatic Inhaler

(71) Applicant: Keith Alan Roberts, Melbourne, FL (US)

(72) Inventor: Keith Alan Roberts, Melbourne, FL (US)

(21) Appl. No.: 18/739,862

(22) Filed: Jun. 11, 2024

(51) Int. Cl. A61M 15/00; A61K 9/00; A61K 31/485

(52) U.S. Cl. CPC A61M 15/009; A61K 9/0009; A61K 9/0078; A61K 31/485; A61M 2205/18; A61M 2205/50; A61M 2205/8206; A61M 2209/088; A61M 2230/06; A61M 2230/50

(57) ABSTRACT

The Overdose Reviver Automatic Inhaler is a method to repeatedly administer Narcan solution automatically, intranasally. When heartbeat sensors relay life-threatening low heartbeat data to a microprocessor chip the programmed functions are activated. Automatically administering atomized, pressurized Narcan solution into nostrils of an unconscious, opioid overdosed person once every two to three minutes and alert for help. The Automatic Inhaler is battery powered, automated, and mechanical. It is worn strapped to a user's neck, a nasal airline with nasal prongs, is secured in place under a user's nose via head, and nasal airline elastic straps. An electromagnetic solenoid depresses an air release valve on a pressurized air canister containing enough Narcan solution and pressurized air for at least ten one second squirts. A precautionary preemptive action for someone who knowingly is using opioids, most likely home alone. It's a method to revive oneself in case a lethal dose is ingested.

### BACKGROUND OF THE INVENTION

Opioids, and especially fentanyl overdose deaths, is an out-of-control epidemic. Wherein

the U.S. is being saturated with fentanyl. Wherein last year in 2023 The U.S. recorded over 100,000 opioid overdose deaths, wherein over 60,000 people have been revived with Narcan. Wherein someone who is using opioids is at risk of receiving a lethal dose of fentanyl and death.

Wherein said Narcan is an opioid overdose reversal agent administered intranasally. Wherein a currently available Narcan nasal administering method is a one-time use, single dose, disposable dispenser. Wherein a nasal tip is inserted into a nostril, wherein it requires two fingertips, and a thumb to depress a plunger wherein releasing Narcan solution. Wherein said Narcan nasal dispenser works great if a responder is around in time to administer said Narcan solution. Wherein mostly everyone who has died from said opioid overdose did not receive said Narcan.

Wherein if a person falls unconscious due to said opioid overdose, and no said responder is around to administer said Narcan, wherein said person could die. Wherein said unconscious, opioid overdosed person cannot administer their own said Narcan, or can they. Whereas The Overdose Reviver Automatic Inhaler is designed and programmed to automatically administer said Narcan solution intranasally without assistance from said responder, same as setting an alarm clock to awake, wherein said Automatic Inhaler is setting a life-threatening low heart rate alarm to awake with said atomized, pressurized Narcan solution automatically administered intranasally.

The said Automatic Inhaler is a method to repeatedly, automatically administer said atomized, and said pressurized Narcan solution in a liquid form intranasally. Wherein said Automatic Inhaler is worn strapped to a user's neck via a neck strap, wherein a nasal airline with nasal prongs, is secured in place under said user's nose via head, and nasal airline elastic straps with adjustable snaps.

Wherein said Automatic Inhaler is battery powered, mechanical, and automated. Wherein heartbeat sensors relay life-threatening low heart rate data to a microprocessor chip, wherein said microprocessor chip activates the electrical and mechanical programmed functions, whereby automatically administering said atomized,

and said pressurized Narcan solution through said nasal airline with nasal prongs to said user's nostrils once every two to three minutes and alerts for help.

Wherein said Automatic Inhaler is intended for someone who knowingly is using opioids, most likely home alone, therefore taking a precautionary preemptive action to automatically revive themself wherein a lethal dose could be ingested.

### SUMMARY OF THE INVENTION

Keeping up with the changing of times, wherein to help combat said opioid overdose deaths. Wherein said Automatic Inhaler is a last line of defense, wherein said unconscious, opioid overdosed person can be their own said responder wherein their life could be rapidly slipping away.

Wherein said Narcan solution and said pressurized air is supplied by The Overdose Reviver Pressurized Spray. Wherein a small pressurized air canister consisting of; a valve covering top cap, an atomizing air release valve, a 3″ long extension spray tube, a tubular shaped said pressurized air canister, wherein the dimensions are approximately ⅞″ width×4″ length. Wherein said Pressurized Spray application number is 18/741050.

Wherein said pressurized air canister provides two different methods of administering said Narcan solution intranasally. Wherein using said pressurized air canister as is, with or without the said 3″ long extension spray tube. Wherein said responder can aim from the base of a person's nostrils, whereby depressing a valve opening push needle on said

atomizing air release valve, wherein said responder can administer said atomized, pressurized Narcan solution into nostrils of said unconscious opioid overdosed person. Or whereby removing said air release valve, wherein inserting said pressurized air canister into said Automatic Inhaler, whereby supplying the necessary said pressurized air, and said Narcan solution for said Automatic Inhaler to perform an automatic reviving method.

Wherein using a calculating formula to detect a life-threatening low heart rate, wherein two heartbeats one second apart is 60 BPM. Wherein two heartbeats two seconds apart is half 30 BPM. Wherein a heart monitor sensor and a heartbeat sensor relays heart rate data to a microprocessor chip, wherein said microprocessor chip calculates said heartbeat data as seconds, wherein said microprocessor chip has a stopwatch seconds counting alarm clock with a speaker, wherein said alarm clock setting activation is for two consecutive said heart monitor seconds data. Wherein said heart monitor seconds data and said heartbeat seconds data are synchronized exactly one second apart, wherein said heart monitor seconds data and said heartbeat seconds data alternates exactly one second apart. Wherein said heart monitor seconds data is detected, said stopwatch seconds counting alarm clock starts counting, wherein if said heartbeat seconds data is detected before said heart monitor seconds data counts two consecutive said heart monitor seconds data, said heart monitor seconds data will reset to zero, same as pressing a snooze button on an alarm clock. This cycle will repeat continuously until said heart monitor seconds data is not reset back to zero, wherein two consecutive said heart monitor seconds data is counted. Wherein two consecutive said heart monitor seconds are two seconds apart, wherein said heart rate equates to 30 BPM. Wherein 30 BPM is a life-threatening low heart rate, wherein time to activate said automatic reviving method. Wherein too much Narcan is not Problematic whereas zero Narcan could be fatal. Wherein Narcan will not affect someone who does not have opioids in their system.

### BRIEF DESCRIPTION OF THE DRAWINGS

FIG. 1 is a cutaway side view of the preferred embodiment, and inner components.

FIG. 2 is a side view of the preferred embodiment, and exterior features.

FIG. 3 is a side view, a profile of the embodiment with head, neck, and nasal airline elastic straps with adjustable snaps.

FIG. 4 is a side view, a profile of the preferred embodiment strapped to FIG. 3 with its nasal airline connected to the preferred embodiment's air outlet port.

FIG. 5 is a perspective view, the embodiment is a pressurized air canister with Narcan solution.

FIG. 6 is a perspective view, the embodiment is a currently available Narcan solution nasal administering dispenser.

FIG. 7 is a sectional view, the embodiment is a silhouette, a partial profile of an adult face, forehead, nose, lips and chin. FIG. 6 and FIG. 7 is simulating the placement to administer a currently available Narcan solution nasal dispenser.

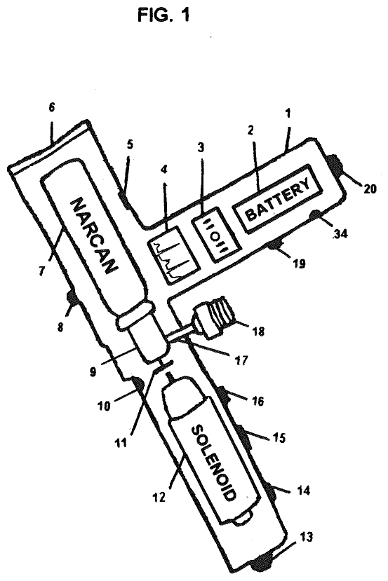

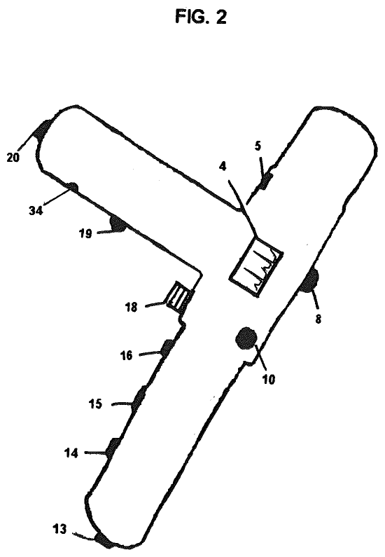

### DETAILED DESCRIPTION OF THE PREFERRED EMBODIMENTS

The Overdose Reviver Automatic Inhaler is described herein as “Automatic Inhaler.” The housing body of said Automatic Inhaler 1 is constructed of a formed moldable material, wherein the dimensions are approximately 1½″ housing body in a tee shaped configuration wherein the dimensions are approximately 6″ height×9″ length.

Wherein said Automatic Inhaler 1 is turned on 13 the electric circuitry and a microprocessor chip 3 is turned on in a standby mode, wherein a stopwatch seconds counting alarm clock with an open switch is turned on in a snooze mode, wherein said microprocessor chip 3 receives and calculates a heart monitor sensor 4, data and a heartbeat sensor 8, data thereby detecting a life-threatening low heart rate.

Wherein there is a three second delay to power on the programmed functions, whereby the said three second delay in time gives a user notice that said Automatic Inhaler 1 is about to activate, allowing said user time to turn off said Automatic Inhaler 13. If said Automatic Inhaler 1 is removed before turning it off 13, said microprocessor chip 3 will detect zero heartbeats, therefore delaying activation of the said programmed functions, thereby allowing said microprocessor chip 3 time to recognize if there is an instant change in temperature sensor data, to help prevent a false alarm.

Wherein if said microprocessor chip 3 detects said life-threatening low heart rate said open switch on said stopwatch seconds counting alarm clock closes sending power to said electromagnetic solenoid 12, depressing a valve opening push needle 11 on an atomizing air release valve 9 on a pressurized air canister 7 containing Narcan solution wherein automatically administering atomized, pressurized Narcan solution through a nasal airline with nasal prongs 24 to said user's nostrils once every two to three minutes, and alert for help.

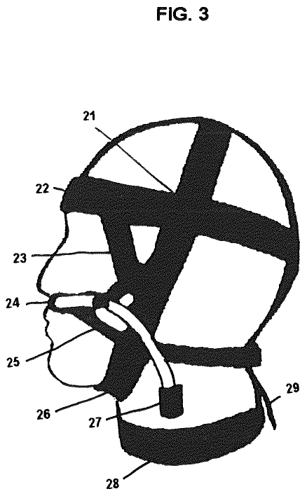

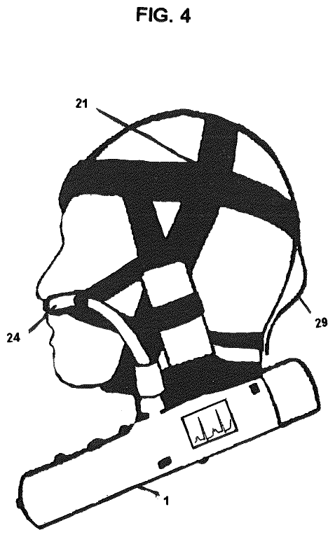

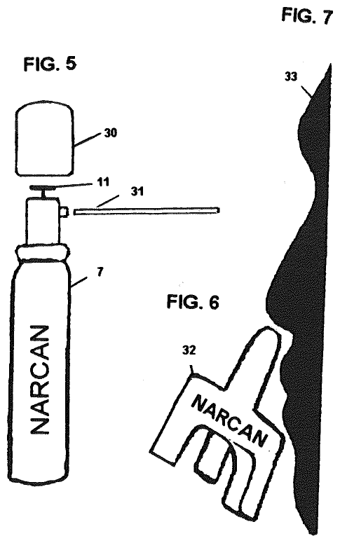

Said pressurized air canister 7 contains enough said Narcan solution, and said pressurized air for at least ten one second 4 mg squirts of said Narcan solution. Wherein said pressurized air canister 7 is tubular shaped, wherein the dimensions are approximately ⅞″ width×4″ length. Wherein said empty pressurized air canister 7 can be removed and replaced through said Automatic Inhalers 1 end cap 6. Wherein before replacing said empty air canister 7, wherein said atomizing air release valve 9 needs to be removed from said pressurized air canister 7. Whereby removing and replacing said empty air canister 7 wherein a numerical digit counting display screen 34 resets to zero,

wherein said numerical digit counter registers one digit every time said pressurized air canister 7 is activated, wherein same as a camera counts pictures taken, Therefore said user knows how many times said pressurized air canister 7 has been activated, whereby said user can judge when said pressurized air canister 7 needs to be replaced.

Wherein said atomizing air release valve 9, a 12 volt dc battery 2, a charging port 5, a rubber airline tube 17 is disposed within the inside of said Automatic Inhalers 1 said housing body. Wherein said rubber airline tube 17 connects said atomizing air release valve 9 to said Automatic Inhalers 1 said air outlet port 18.

Wherein a LED light indicates when said Automatic Inhalers 1 said electric circuitry is turned on. Wherein a speaker to communicate with 911 15, wherein a push button to call 911 16, wherein a display screen illuminates green and continuously strobes to a heartbeat rhythm, wherein if said heart rate drops to two heartbeats two seconds apart, thereby said display screen illuminates red and strobes to said heartbeat rhythm. Wherein GPS location is activated, wherein said alarm speaker thereby continuously squelches an SOS signal, wherein a red LED light thereby continuously strobes an SOS signal.

Wherein said Automatic Inhaler 1 is activated, wherein said programmed functions thereby continues until said Automatic Inhaler 1 is turned off 13, or said battery 2 runs too low, wherein a test button 20 works independent of said Automatic Inhalers 1 said on/off button 13, whereby depressing said test button 20 said user can look through a cutout viewing window 10, covered over with clear plastic located at the position wherein said electromagnetic solenoid 12 depress a valve opening push needle 11 disposed inside of said atomizing air release valve 9 on said pressurized air canister 7 wherein said user can see for themself that said electromagnetic solenoid 12 depresses said valve opening push needle 11.

Wherein said Automatic Inhaler 1 is strapped to said user's neck via elastic neck straps 28, wherein a nasal airline with nasal prongs 24 is strapped and secured in place under said user's nose with head, and nasal airline elastic straps 25, said nasal airline with nasal prongs 24 connects to said Automatic Inhalers 1 said air outlet port 18 with a connection fitting 27, embodiment FIG. 3 consisting of; a forehead strap wherein circles around said user's head, a support strap 23, helps to secure said forehead strap 22 to a chin strap 26, wherein said chin strap circles from under said user's chin to around the top of said user's head, wherein said nasal airline elastic strap 25, runs from under said user's nose and circles around to the back of said user's neck, wherein a middle strap 29 runs from the middle of said user's forehead, up to the middle crown, down the middle backside, wherein hangs down long enough to connect with said elastic neck strap 28, making it difficult to pull off or dislocate said head, and said nasal airline elastic straps.

Wherein FIG. 5 is a perspective view, wherein an embodiment is said pressurized air canister 7 containing said Narcan solution, a valve covering top cap 30, a 3″ long extension spray tube 31, said valve opening push needle 11 disposed inside of said atomizing air release valve 9, wherein said pressurized air canister 7, can be used as is, wherein a responder can aim from the base of said unconscious opioid overdosed person's nostrils, wherein depressing said valve opening push needle 11 whereby administering said atomized, pressurized Narcan solution into nostrils of said opioid overdosed person, wherein embodiment FIG. 6. is a perspective view of said currently available Narcan solution nasal administering dispenser 32, wherein embodiment FIG. 7 is a side and sectional view, a silhouette, a profile of an adult face, forehead, nose, lips and chin 33, wherein FIG. 6 and FIG. 7 is simulating the placement to administer said currently available Narcan solution nasal administering dispenser.

What is claimed:

1. The Overdose Reviver Automatic Inhaler is a method to automatically repeatedly administer atomized pressurized Narcan solution intranasally to an unconscious opioid overdosed person every two to three minutes and alert for help consisting of:

2. GPS a red LED light and a speaker continuously strobes and squelches an SOS signal.

3. The Automatic inhaler's housing body is worn strapped to a user's neck via neck straps wherein a nasal airline with nasal prongs is secured under a user's nose via head and nasal airline elastic straps with adjustable snaps.

4. A microprocessor chip receives and converts life-threatening low heartbeat sensors data into countable seconds whereby a stopwatch seconds counting alarm clock's open switch closes therefore a battery powers on the electrical and mechanical programmed functions.

5. An independent test switch when depressed a user can look through a cutout viewing window covered with clear plastic to verify an electromagnetic solenoid depresses an air release valve on a pressurized air cannister containing Narcan solution and the composition passes through a nasal airline with nasal prongs.

6. An independent switch and speaker a user can directly call and communicate with a 911 operator.

7. A numerical digit consecutive counting pressurized air canister activation dislay screen.
